(19) United States
(12) Patent Application Publication
(10) Pub. No.: US 2024/0293263 A1
(43) Pub. Date: Sep. 5, 2024

(54) APPARATUS AND METHOD FOR PROTECTING SURGICAL AND IMPLANT SITES

(71) Applicants: Roger W. Little, Salt Lake City, UT (US); Heather Pickett, Salt Lake City, UT (US)

(72) Inventors: Roger W. Little, Salt Lake City, UT (US); Heather Pickett, Salt Lake City, UT (US)

(21) Appl. No.: 18/116,969

(22) Filed: Mar. 3, 2023

(51) Int. Cl. A61F 13/02; A61L 15/58

(52) U.S. Cl. CPC A61F 13/0246; A61L 15/58

(57) ABSTRACT

Disclosed are devices and methods for protecting and sealing sites of, for example, PICC lines, medical implants with external components, surgical sites, or other wounds. In some embodiments, disclosed devices include a roll/rod having a handle portion and a receiving portion, and a covering or wrap disposed on the receiving portion. The handle portion is configured to be grasped by a user while covering their PICC line, medical implant, surgical site, or wound with the wrap. The wrap can be a plastic cling wrap, such as those used to protect food or to secure objects to a pallet or other substrate. Such wraps can be made from polyvinyl chloride (PVC), low-density polyethylene (LDPE), polyvinylidene chloride (PVDC), polyester (PE), and the like.

### BACKGROUND

### Technical Field

This disclosure generally relates to the sealing and protection of surgical and implant sites using stretch plastic.

### Related Technology

Each year, about 650,000 cancer patients receive chemotherapy in outpatient oncology clinics in the United States. Patients receiving chemotherapy are at risk for developing infections that may lead to hospitalization, disruptions in chemotherapy schedules, and even death. Generally, patients receiving multiple, repeated rounds of chemotherapy are provided with a peripherally inserted central catheter, also called a PICC line.

A PICC line is a long, thin tube that is typically inserted through a vein in the arm and passed through to the larger veins near the heart. The PICC line provides access to the large central veins near the heart and is generally used to provide the patient with medications or liquid nutrition. A PICC line also allows for hook ups to intravenous lines (IVs) and other PICC lines, among other things.

A PICC line is generally intended to be temporary and requires careful care and monitoring. Common PICC line complications can include bleeding, nerve injury, irregular heartbeats, damage to veins in the arm, blood clots, infection, and/or a blocked or broken PICC line.

PICC lines and surrounding skin and underlying tissue should be kept dry to protect the functioning of the PICC line and to prevent infection. This means that patients equipped with a PICC line need to cover or otherwise seal the PICC line prior to engaging in water-based activities, such as bathing or showering. Current practice to protect PICC lines is to provide patients with a square sheet of plastic configured with double sided tape. Patients are instructed to apply (e.g., wrap) this square sheet of plastic around their limb to protect the PICC line(s).

However, wrapping a square sheet of plastic around an arm of non-uniform diameter may causes the plastic to wrinkle. Such wrinkling may result in a poor fit over the PICC line and may result in discomfort for the patient. Additionally, when wrinkling occurs in the double-sided tape, the tape may not always able to adhere securely to the patient's arm and skin, further causing a poor and uncomfortable fit. Wrinkling of the plastic can provide gaps that permit ingress of water, microbes and other contaminants under the plastic, which can come into contact with the PICC line, its components, and the associated wound in the patient's limb, potentially defeating the purpose of the plastic covering. The plastic may in fact collect rather than exclude contaminants and infectious agents.

Another issue occurs when removing the sheet of plastic from regions that have fresh wounds, scarring, and scabbing. The tape can adhere to hair, sensitive wounds, and scabs, causing pain and discomfort to the patient. Thus, although sheets of plastic can be provided to patients with PICC lines or other wounds that require a dressing and need to be waterproofed, they may cause more harm and discomfort than they prevent. It short, it is important for a protective covering be temporary, easy to apply, and easy to remove to avoid agitating or harming the wound.

Thus, what is needed is a temporary, waterproof covering for protecting PICC lines, wounds, incisions, medical implants, and the like that is easy to apply, that provides a sound and secure barrier to moisture and contaminants, and which is easy to remove without causing pain and harm to the patient.

### SUMMARY

Embodiments of the present disclosure are directed to devices and methods for protecting and sealing PICC lines, medical implants with external components, surgical sites, and other wounds. In some embodiments, the disclosed devices may include a roll or rod having a handle portion, a receiving portion, and a roll of cling wrap on the receiving portion. The handle portion is configured to be grasped by a user to facilitate placing the cling wrap around the arm, other limb, or other site of a PICC line, medical implant, surgical site, or wound.

Also disclosed are methods for protecting and sealing PICC lines, skin and adjacent tissue, medical implants with external components, surgical sites, and wounds. In some embodiments, the methods include placing or wrapping a protective layer of plastic cling wrap at or around a site, which can be a PICC line, medical implant with external components, surgical site, or other wound. The protective wrap may be disposed on a receiving portion of a roll, where the roll also includes a handle portion. The protective wrap is configured to be waterproof, thereby protecting and sealing the site of application.

In some embodiments, the protective layer or covering is configured to be temporarily applied to the site (i.e., skin and tissue at the PICC line, medical implant with external components, surgical site, or other wound). The protective layer is configured to gently cling to skin, medical equipment to be protected, and to itself in order to reliably seal and exclude moisture and contaminants. In some embodiments, the protective layer is configured to cling and adhere to itself in order to seal and exclude moisture and contaminants without the use of tape or other adhesives. The protective layer may be configured to adhere to itself more it adheres to skin and hair so that it can be removed from the patient without pulling or damaging sensitive skin, tissue wounds, the PICC line, or medical implant at the site to be protected.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 is a side perspective view that schematically illustrates an embodiment of protective layer roll; and

FIG. 2 is a top perspective view of the protective layer roll of FIG. 1.

### DETAILED DESCRIPTION

Embodiments of the present disclosure are directed to devices and methods for protecting and sealing surgical or wound sites of, for example, PICC lines, medical implants with external components, and/or surgical or other wounds. In some embodiments, disclosed devices include a roll/rod having a handle portion, a receiving portion, and a roll of covering or cling wrap disposed on the receiving portion. The handle portion is configured to be grasped by a user to facilitate placing the cling wrap around the arm, other limb, or other site of a PICC line, medical implant, surgical site, or wound.

Also disclosed are methods for protecting and sealing surgical or wound sites, such as the location of PICC lines, medical implants with external components, surgical sites, or other wounds. In some embodiments, the methods include placing or wrapping a protective layer of plastic cling wrap at or around a site, which can be a PICC line, medical implant with external components, surgical site, or other wound. The protective wrap may be disposed on a receiving portion of a roll, where the roll also includes a handle portion. The protective wrap is configured to be waterproof, thereby protecting and sealing the site of application.

In some embodiments, the protective layer or covering is configured to be temporarily applied to the site (i.e., skin and tissue at the PICC line, medical implant with external components, surgical site, or other wound). The protective layer is configured to gently cling to skin, medical equipment to be protected, and to itself in order to reliably seal and exclude moisture and contaminants. In some embodiments, the protective layer is configured to cling and adhere to itself in order to seal and exclude moisture and contaminants without the use of tape or other adhesives. The protective layer may advantageously be configured to adhere to itself more it adheres to skin and hair so that it can be removed from the patient without pulling or damaging sensitive skin, tissue wounds, the PICC line, or medical implant at the site to be protected.

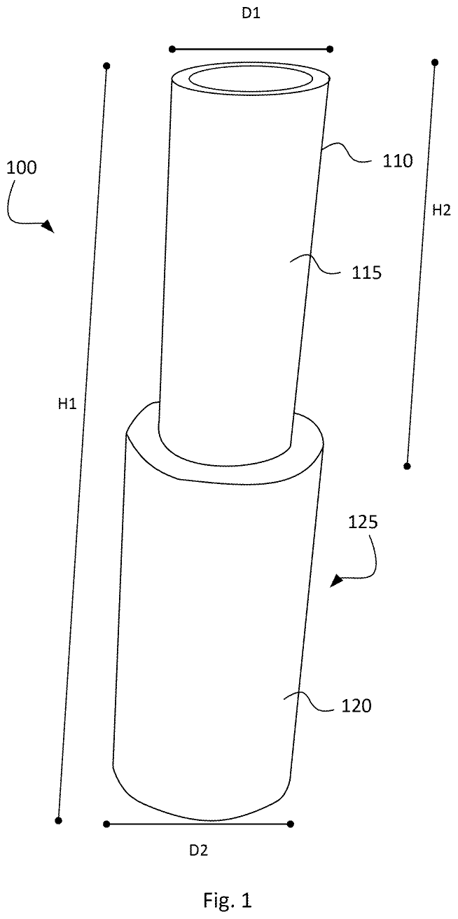

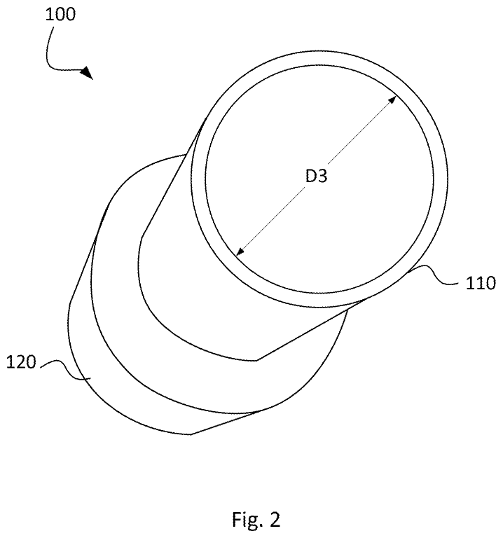

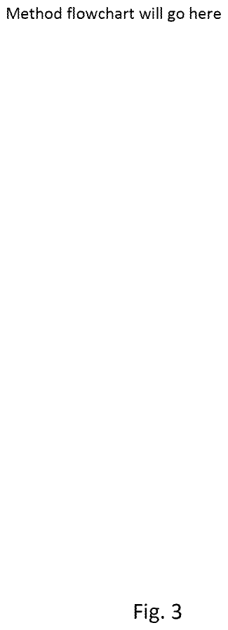

Reference is now made to the drawings. FIG. 1 is a side perspective view that schematically illustrates an example protective layer roll 100 and FIG. 2 is a top perspective view thereof. The protective layer roll 100 includes a rod 110 having a handle portion 115 at one end, which are attached to a receiving portion 125 at an opposite end that includes a covering material 120. The covering sheet 120 is advantageously wrapped around and received by the receiving portion 125 and can be easily unwrapped when placing the sheet 120 at a desired site to be protected. In some embodiments, the receiving portion 125 can be configured to rotate independent of the handle 110. In other cases, they can be rigidly attached without permitting independent rotation.

The handle portion 115 of the rod 110 is configured to be grasped by a user to facilitate applying the covering 120 to a site, such as around skin and tissue where a PICC line has been placed, a medical implant with external components, a surgical site, or other wound. The roll 100 can have a height H1 in a range of about 7 inches to about 14 inches, preferably about 9 to about 12 inches, such as about 8 inches, 11 inches, 11.5 inches, or 12.5 inches, or a height within a range defined by any two of the foregoing values. The handle portion 115 can have a height H2 in a range of about 3 to about 7 includes, preferably about 4 to about 6 inches, such as about 3.5 inches, 4.5 inches, 5 inches, or 5.5 inches, or a height within a range defined by any two of the foregoing values. In some embodiments, the height H2 of the handle portion 115 can be approximately half the height H1 of the roll 100 such that the receiving portion 125 and wrap 120 can have a height that is approximately equal to the height of the handle portion. Alternatively, depending on the size of the area to be protected, the wrap 120 can have a height that is less than or greater than the height of the handle 115, such as 50%, 60%, 70%, 80%, 90%, 95%, 105%, 110%, 120%, 130%, 140% or 150% of the height of the handle 115.

In some embodiments, the receiving portion 125 can be grooved or otherwise recessed compared to the handle portion 115. Grooving or recessing the receiving portion 125 can allow the covering 120 to nest within the groove or recess and be securely held by the receiving portion 125. In some embodiments, the handle portion 115 can be textured to improve a user's grip of the handle portion 115.

The rod 110 can have a diameter D1 in a range of about 0.75 inch to about 3 inches, preferably about 1 inch to about 2.5 inches, such as about 1.5 inch, 2 inches, or a diameter within a range defined by any two of the foregoing values. As illustrated, the rod 110 can be hollow; with a lumen running between the handle portion 115 and the receiving portion 125, to reduce weight. In such cases, the rod 110 has an inner diameter D3. In some embodiments, D3 is therefore substantially the same as D1, less the sum of wall thickness on both sides of the rod 110. In some embodiments, D3 may be about 0.25 inch to about 0.75 inch, such as 0.5 inch, less than D1. Beneficially, a hollow rod 110 is lighter than a solid rod, meaning a user can more easily grasp and control the protective layer roll 100. For example, if the user is a child, the roll 100 is advantageously light and easy to manage when applying the covering 120 to a site.

The covering 120 can have a diameter D2 of about 2 to about 8 inches, preferably about 2.5 to about 7 inches, such as about 3 inches, 4 inches, 5 inches, 5.5 inches, 6 inches, or a diameter within a range defined by any two of the foregoing values. The diameter of the covering 120 will largely depend on whether and how much the device has been unused, with the diameter being at a maximum before use, at a minimum when fully used up, or in between after being partially used.

In some cases the size of the device 100 may vary depending on the user. For example, if the user of the roll 100 is a child, the diameter D2 of the covering 120 can be smaller, e.g., about 3 to 4.5 inches. If the user of the roll is an adult, the diameter D2 of the covering 120 can be larger, e.g., about 5 to 6.5 inches. Beneficially, the protective layer roll 100 can be used by adults and children to easily cover and protect the site (e.g., the site of a PICC line, medical implant with external components, IV hook up, surgical site, other wounds, and the like).

The rod 110 can be constructed from cardboard and/or rigid cardstock. Additionally, and or alternatively, the rod 110 can be constructed from regid or semi-rigid plastic. The rod 110 should be sufficiently rigid and robust to be grasped and manipulated by a user without bending, collapsing, breaking, or otherwise failing. The rod 110 should also be sufficiently light to be easily handled and controlled by a user.

The covering 120 can be a stretch plastic, plastic wrap, cling wrap, or plastic film. In some embodiments, the covering 120 is wrapped directly around the receiving portion 125. In some embodiments, the covering 120 can be a pre-existing roll that is placed over the receiving portion 125. In either case, the covering 120 is advantageously replaceable. Either another pre-existing roll of covering 120 can be slid onto the receiving portion 125 or new covering 120 can be wrapped around the receiving portion 125. In either case, a user may only need one rod 110 and can re-supply the covering 120. The amount of covering 120 may allow for approximately 10-25 uses or wraps before needing a replacement. However, single use devices can also be provided, such as in a hospital for one-time use to preserve sterility.

Examples of wraps that can be used in the present invention include, but are not limited to, cling plastic wrap for storing food (e.g., Saran wrap or Glad food wrap). Such plastic wraps are typically made from polyvinyl chloride (PVC) or low-density polyethylene (LDPE), two long polymers with tightly bound and coiled chains of molecules. The tight molecular bond and spring-like quality of the chains are why plastic wrap made from these polymers can stick really well to itself and other objects and provides an excellent job of blocking moisture and odors. Industrial cling wraps, such as those used to secure goods onto pallets or other support structures can be made from polyvinylidene chloride (PVDC). Polyester (PE) films are another example. Other films may include a blend of wax and a thermoplastic polymer (e.g., a thermoplastic elastomer) order to be plastically deformable yet thermally stable at room or body temperature.

The device that includes the protective covering may advantageously be stored in a sterile container prior to use and placed back into the sterile container between uses. The sterile container can be a can, a tube, a bag, or other appropriate container that can be opened and resealed as needed.

Also disclosed are methods of applying a covering or protective layer to a wound, PICC line, IV, or surgical site. The methods can include providing a device that contains a protective layer or covering (see FIGS. 1 and 2) and wrapping the protective layer or covering around the site by grasping the handle. The site may be a site of a medical implant with external components, a PICC line, an IV hook up, and/or a surgical or other open wound, such as a fresh tattoo.

Wrapping the protective layer around the site can include grasping the handle portion 115 of the roll 100 from FIGS. 1 and 2. While grasping the handle portion 115, a portion of the covering 120 will be wrapped around the site. For example, if the site is located on an upper extremity, a portion of the covering 120 will be wrapped around the upper extremity. The portion of the covering 120 may be wrapped around the upper extremity more than once, such that portions of the covering 120 overlap with each other. The overlapping portions of the covering 120 advantageously self-adhere, without requiring tape or adhesive, thereby reliably sealing the site and protecting the skin, tissue and medical device from water and contaminants. The number of wrappings or overlapping layers can be selected to provide a desired level of protection, typically at least two wrappings or overlapping layers, and preferably at least three, at least four, or at least five.

The covering 120 can be a stretch plastic, cling plastic wrap, plastic film, or other suitable waterproof material (e.g., PVC, LDPE or PVDC). The covering 120 is configured to cling and adhere to itself, typically more strongly than it adheres to skin, tissue and hair. The covering 120 is also configured to be easily removable from itself, such that removing the covering 120 from the site does not pull or tear at any components at the site, such as skin, tissue, scabs, and surgical equipment. For example, when the site is a PICC line implantation, removal of the covering 120 will advantageously not pull or tear away the hookups of the PICC line. Further, the covering 120 advantageously does not require external tape or adhesives, so there is no excessive adhesive or stickiness to pull and tear at any components of the site, including skin, hair, sensitive wound tissue, or surgical components. The material of the covering 120 should be easily conformable to and around a round limb, so that it does not wrinkle upon application and doesn't leave any gaps or openings. Of course, even if there are wrinkles or folds, placing several layers of the wrap around the side will provide a water-proof barrier that excludes moisture and contaminants.

Application of the covering 120 temporarily seals the site from water and contaminants. Sealing the site allows patients to safely, comfortably, and easily engage in water activities without worrying about the integrity of the site. For example, patients can apply the covering 120 to the site before taking a bath, showering, or soaking in a hot tub. Because the covering 120 is self-adherent and sticks to itself and also to skin (albeit without undue force), it reliably and securely seals the site. After bathing, the patient can quickly, easily, and painlessly remove the covering 120 from the site and go about the rest of their day. In some embodiments, the covering 120 can be applied over a wound dressing to keeps the dressing dry and clean throughout the day. The wrap may be kept in place for as long as may be desired. However, it should be removed periodically to permit the skin and tissue to breath.

The covering 120 may be used to seal and protect sites such as a medical implant or PICC line site, surgical wound, or other area requiring protection. For example, the covering 120 can be used to seal and protect open or fresh wounds, such as cuts, scrapes, lacerations, blisters, and fresh tattoos. The covering 120 may also be used to attach ice or other therapeutic packs to a limb of the body. As the covering 120 can be a plastic wrap or film, the covering 120 is particularly adapted for attaching things to a curved profile, such as an arm or leg.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. A device for protecting a medical implant with external components at a site on a patient's body, the device comprising:
a rod having a handle portion configured to be gripped by a user and a receiving portion; and
a plastic covering disposed on the receiving portion, wherein the plastic covering is configured to cover a site on the patient's body, the plastic covering protecting the site and preventing the site from moisture and contaminants.

2. The device of claim 1, wherein the receiving portion is rotatably connected to the handle portion.

3. The device of claim 1, wherein the receiving portion is non-rotatably connected to the handle portion.

4. The device of claim 1, wherein the plastic covering is configured to adhere to itself without external tape or adhesive.

5. The device of claim 1, wherein the plastic covering is configured to lightly adhere to and seal against skin without pulling off skin, tissue, hair or medical components upon removing the covering from the site.

6. The device of claim 1, wherein the plastic covering comprises plastic cling wrap.

7. The device of claim 6, wherein the plastic cling wrap comprises at least one of polyvinyl chloride (PVC), low-density polyethylene (LDPE), polyvinylidene chloride (PVDC), or polyester (PE).

8. The device of claim 1, wherein the medical implant having external components comprises a PICC or IV line.

9. The device of claim 1, wherein the device has an overall height in a range of about 7 inches to about 14 inches, and the handle portion has a height in a range of about 3 inches to about 7 inches.

10. A method for protecting a surgical or wound site on a patient's body, comprising:
wrapping a protective plastic covering around the surgical or wound site; and
ensuring the protective plastic covering has adhered to itself and skin at the surgical or wound site,
wherein the protective plastic covering is configured to temporarily seal and protect the surgical or wound site from moisture and contaminants, and wherein the protective plastic covering adheres to itself without external tape or adhesive.

11. The method of claim 10, wherein the surgical or wound site includes a medical implant emerging from the patient's skin and wherein the medical implant includes external components.

12. The method of claim 11, wherein the medical implant comprises a PICC line.

13. The method of claim 11, wherein the medical implant comprises an IV line.

14. The method of claim 10, wherein the surgical or wound site includes an open wound.

15. The method of claim 10, wherein the protective plastic covering is wrapped at least 2 times around the surgical or wound site.

16. The method of claim 10, wherein the protective plastic covering is wrapped at least 3 times around the surgical or wound site.

17. The method of claim 10, wherein the protective plastic covering is wrapped at least 4 times around the surgical or wound site.

18. The method of claim 10, wherein the protective plastic covering is provided by a device comprising a rod having a handle portion configured to be gripped by a user, a receiving portion, and a plastic covering disposed on the receiving portion.

19. The method of claim 18, wherein the device is stored in a protective container prior to use, the method further comprising removing the device from the protective container and applying the protective plastic covering to the surgical site or wound.

20. The method of claim 19, further comprising placing an unuse portion of the device back into the protective container prior to a subsequent use.
